(19) United States
(12) Patent Application Publication
(10) Pub. No.: US 2025/0259712 A1
(43) Pub. Date: Aug. 14, 2025

(54) METHOD AND SYSTEM FOR OBTAINING, CONTROLLING, ACCESSING AND/OR DISPLAYING GENETIC IDENTIFICATION INFORMATION OF PLANTS, NON-HUMAN

(71) Applicants: Duangkamol STROHL, Lewes, DE (US); Paul McLAREN, Lewes, DE (US); Julie RENFROE, Lewes, DE (US)

(72) Inventors: Duangkamol STROHL, Lewes, DE (US); Paul McLAREN, Lewes, DE (US); Julie RENFROE, Lewes, DE (US)

(73) Assignee: STR-ID, Inc., Lewes, DE (US)

(21) Appl. No.: 19/045,568

(22) Filed: Feb. 5, 2025

Related U.S. Application Data
(60) Provisional application No. 63/553,582, filed on Feb. 14, 2024.

(51) Int. Cl. G16B 50/40; C12Q 1/6806; C12Q 1/6869; G16B 45/00; H04L 9/00

(52) U.S. Cl. CPC G16B 50/40; C12Q 1/6806; C12Q 1/6869; G16B 45/00; H04L 9/50

(57) ABSTRACT

A method and system for obtaining and controlling non-human genetic identification information are disclosed. The method includes providing identifying information of a plant, a non-human animal, or a living plant or animal product to a secure website using an electronic communication device; taking a genetic material-containing sample from the plant, animal or living product; providing the genetic material-containing sample to a genetic material analysis facility; analyzing the genetic material at a plurality of loci to produce a genetic identity for the plant, animal or living product; recording the identifying information and the genetic identity in a blockchain ledger; and enabling a user to display on an electronic communication device a code corresponding to the genetic identity. The system includes a genetic material sampling kit, an optional DNA analysis kit, and electronic communication device(s) configured to enter the plant's, animal's or living product's identification information, record the identification information and the genetic identity in the blockchain ledger, and display a code corresponding to the genetic identity.

### RELATED APPLICATION(S)

This application claims priority to U.S. Provisional Pat. Appl. No. 63/553,582, filed Feb. 14, 2024, incorporated herein by reference.

### FIELD OF THE INVENTION

The present invention generally relates to the field(s) of obtaining, controlling and accessing genetic identification information. More specifically, embodiments of the present invention pertain to novel methods and systems for obtaining, storing, controlling and accessing non-human genetic identification information, particularly using DNA analysis and blockchain data/transaction storage and retrieval.

### DISCUSSION OF THE BACKGROUND

Animal identification (ID) generally relies on records of individual farm animals or groups of farm animals so that they can be easily tracked from their birth through the marketing chain. Historically, animal ID was used to indicate ownership and prevent theft, but the reasons for identifying and tracking animals have evolved to include rapid response to animal health and/or food safety concerns. The U.S. Department of Agriculture (USDA) has proposed an approach for managing farm animal identification (known as Animal Disease Traceability) that allows individual states and tribal nations to choose their own degree of in-state animal identification and traceability for livestock populations. However, the USDA requires that all animals moving in interstate commerce have a form of ID that allows traceability back to their originating state or tribal nation. In 2013, the USDA published a final rule (9 CFR, part 86) that establishes requirements for the identification of livestock and documentation for certain interstate movements of livestock.

Nonetheless, there are a number of shortcomings in the USDA rules. For example, many cattle are exempt from the rule and do not need official identification to travel intrastate or interstate. In addition, pertinent records and data are sometimes erroneous, and often cannot be located. For example, in 2021-2022, visual metal National Uniform Ear tagging System (NUES) tags represented about a third of all traces and 70% of terminated traces. With visual metal ear tags, the animal must be restrained to allow the ear tag to be read and transcribed. Visual ear tag numbers are typically recorded on paper or manually entered in a database, leading to reading and transcription errors. Electronic ear tags has reduced such errors, although ear tag scanning errors and electronic failures can still occur. In either case, when the animal dies, the tag is frequently not maintained with or otherwise correlated to the animal, leading to a loss of traceability at a time when it may be needed most. With breeding livestock and racehorses, which can have a significantly higher commercial value than livestock used for other purposes, erroneous or false identification of individual animals can lead to false insurance and lineage/parentage claims, incorrect registrations, and lack of proper traceability.

One state-of-the-art approach for reliably identifying unique non-human animals is microchipping, or inserting a readable microchip under the skin of the animal. Microchipping has some drawbacks, however. It is invasive, the microchip has an associated cost, and under certain conditions (e.g., in the presence of a sufficiently large magnetic field), the data on the microchip can be scrambled. Verification of the animal depends completely on correct human insertion of the microchip identifying the animal into the animal identified by the microchip.

Certain commercially valuable plant and animal products can be copied or “knocked off,” and the copies or knock-offs may not be very different genetically from the authentic products. For example, Densuke melons typically cost around US$250, and in 2008, a Densuke melon sold for roughly US$6,100. However, copies or knock-offs of Densuke melons can be purchased for less than US$10.

The field of wildlife forensic analysis has established standards for forensic analysis of animals (see, e.g., “SWFS Standards and Guidelines for Wildlife Forensic Analysis,” published by the Society for Wildlife Forensic Services [SWFS] Technical Working Group, version 3 of which was published online 19 Nov. 2018 at www.wildlifeforensicscience.org/wp-content/uploads/2018/11/SWFS-Standards-and-Guidelines_Version-3_19-11-18.pdf, and “Wildlife Forensics General Standards,” Standard 019, First Edition, published by ANSI/ASB, available online at chrome-extension://efaidnbmnnnibpcajpcglclefindmkaj/https://www.aafs.org/sites/default/files/media/documents/019_Std_el.pdf). The field of forensic botany has also established standards for forensic analysis of plants (see, e.g., “Standards and Guideline for Forensic Botany Identification,” published by SWFS [2013], available online at www.wildlifeforensicscience.org/wp-content/uploads/2016/07/annex-18-standards-and-guidelines-for-forensic-botany-identification-2.pdf).

Blockchain promises to solve at least some problems with managing and authenticating certain documents and/or events, such as contracts, transactions, and the information associated therewith. A blockchain is a data structure that enables creation of an open, distributed digital ledger that can record transactions between two parties efficiently and in a verifiable and permanent way, and is the technology at the heart of bitcoin and other virtual currencies. The ledger can be shared among a network of independent parties and also be programmed to trigger further transactions automatically, while at the same time providing a high level of immutability.

This “Discussion of the Background” section is provided for background information only. The statements in this “Discussion of the Background” are not an admission that the subject matter disclosed in this “Discussion of the Background” section constitutes prior art to the present disclosure, and no part of this “Discussion of the Background” section may be used as an admission that any part of this application, including this “Discussion of the Background” section, constitutes prior art to the present disclosure.

### SUMMARY OF THE INVENTION

In one aspect, the present invention concerns a method of obtaining and controlling non-human genetic identification information, comprising providing identifying information of a plant, a non-human animal or a living plant or animal product to a secure website using a first electronic communication device; taking a genetic material-containing sample from the plant, the non-human animal, or the living plant or animal product; providing the genetic material-containing sample to a genetic material analysis facility; analyzing the genetic material at a plurality of loci to produce a genetic identity for the plant, the non-human animal, or the living plant or animal product; recording the identifying information and the genetic identity in a blockchain ledger; and enabling a user to display on a second electronic communication device a code corresponding to the genetic identity, wherein the first and second electronic communication devices are the same device or different devices.

In various embodiments, the identifying information may comprise at least two of a name, an owner name, an owner address, and a photograph of the plant, the non-human animal, or the living plant or animal product. In other or further embodiments, the first and second electronic communication devices may be independently selected from a smart phone, a smart watch, a personal computer, a tablet computer, and a work station. In some embodiments, the method may further comprise accessing the code using one of the first and second electronic communication devices.

In certain embodiments, the method may further comprise (i) encrypting the identifying information and the genetic identity of the plant, animal, or living plant or animal product prior to recording the identifying information and the genetic identity in the blockchain ledger, (ii) certifying or confirming that the genetic material has been collected, (iii) certifying or confirming that a sample collector has authority to collect the genetic material, or a combination thereof. In other or further embodiments, the method may further comprise (iv) allowing the user to access entries in the blockchain ledger containing the identifying information and the genetic identity and/or (v) authenticating an identity or the identifying information of the plant, animal, or living plant or animal product using the genetic identity.

In embodiments of the method pertaining to the non-human animal, taking the genetic material-containing sample from the animal may comprise collecting saliva from the animal in a vial or tube, swabbing an inner surface of the animal's mouth or nose, or pricking/puncturing the animal's skin and collecting one or more drops of the animal's blood on a swab or piece of absorbent paper. In various embodiments pertaining to the animal, analyzing the genetic material may comprise extracting DNA from the genetic material, and analyzing the DNA by short tandem repeat (STR) analysis, whole genomic and next generation DNA sequencing, mitochondrial DNA (mtDNA) sequencing, or analysis of single-nucleotide polymorphisms (SNPs). In some examples, the animal is a pet, such as a dog or a cat. Alternatively, the animal may be a livestock animal, such as a bovine, a horse or a sheep.

In embodiments of the method pertaining to the plant, analyzing the genetic material may comprise extracting DNA from the genetic material, and analyzing the DNA by analysis of single-nucleotide polymorphisms (SNPs), genotyping by sequencing (GBS), next-generation sequencing (NGS), polymerase chain reaction (PCR)-based sequencing, or 3^{rd} generation sequencing (TGS). In one example, the plant may be an asexually reproduced plant.

In some embodiments, providing the genetic material-containing sample to the genetic material analysis facility comprises shipping the genetic material-containing sample to the genetic material analysis facility in an envelope, sleeve, tube or box. In other or further embodiments, a registrant may provide the identifying information of the plant, the non-human animal, or the living plant or animal product to the secure website, and the method may further comprise enabling the registrant to authorize one or more third parties to access the code on a third electronic communication device. The third electronic communication device may be identical to, the same as, or different from one or both of the first and second electronic communication devices.

The present invention offers a digital non-human genetic (e.g., DNA-based) identity management system and method, where users control the identification information of registered plants, non-human animals, and living plant and animal products in many cases substantially anywhere and/or substantially at any time. In many embodiments, the invention connects easily and directly to users and their electronic communication devices (e.g., smartphones), and leverages advanced privacy protection, genetic identification technology, and tamper-proof blockchain technology to generate and/or authenticate a unique identification scheme for a plant or non-human animal.

The present invention uses plant and animal DNA to distinguish and/or authenticate that plant's or animal's identity (or to authenticate the living plant or animal product) with substantially irrefutable accuracy, while protecting relevant privacy rights. The present system and method overcome the disadvantages of microchipping, thereby saving time, money and resources for pet owners, animal shelters and relevant governmental authorities, and provides an alternative approach for authenticating and/or confirming the identity and/or source of breeding livestock and asexually reproduced plants. The present method and system also enable facile tracing of plant-and animal-based food products through commercial and transportation channels, in the undesirable event of an outbreak of a food-borne pathogen or other disease, to quickly isolate and remove any contaminated plants, animals or related materials, or to demonstrate that certain plants, animals or related materials are unaffected by the outbreak.

These and other advantages of the present invention will become readily apparent from the detailed description of various embodiments below.

### BRIEF DESCRIPTION OF THE DRAWINGS

FIG. 1 is a flow chart of an exemplary method of obtaining and providing non-human genetic identification information in accordance with one or more embodiments of the present invention.

FIG. 2A is a diagram showing an exemplary smart phone display including genetic identification information in accordance with one or more embodiments of the present invention.

FIG. 2B is a diagram showing various systems and functions in a digital track-and-trace methodology in accordance with one or more embodiments of the present invention.

FIG. 3 is a block diagram showing components of a generic system for obtaining and accessing/providing genetic identification information in accordance with one or more embodiments of the present invention.

FIG. 4 is a diagram of an exemplary blockchain in accordance with one or more embodiments of the present invention.

FIG. 5 is a flow chart illustrating an exemplary method of managing genetic identification information in accordance with one or more embodiments of the present invention

FIG. 6 is a flow chart illustrating an exemplary genetic information recording process and exemplary ledger transactions using a public permission blockchain and blockchain network in accordance with one or more embodiments of the present invention.

FIG. 7 is a block diagram illustrating an exemplary identification and genetic information privacy protection system in accordance with one or more embodiments of the present invention.

FIG. 8 is a block diagram illustrating an exemplary blockchain with a distributed ledger for recording genetic and other identification information, as well as transactions related thereto, in accordance with one or more embodiments of the present invention.

FIG. 9 is a block diagram showing components of an exemplary PC/computer system suitable for use in the present system and method.

### DETAILED DESCRIPTION

Reference will now be made in detail to various embodiments of the invention, examples of which are illustrated in the accompanying drawings. While the invention will be described in conjunction with the following embodiments, it will be understood that the descriptions are not intended to limit the invention to these embodiments. On the contrary, the invention is intended to cover alternatives, modifications and equivalents that may be included within the spirit and scope of the invention. Furthermore, in the following detailed description, numerous specific details are set forth in order to provide a thorough understanding of the present invention. However, it will be readily apparent to one skilled in the art that the present invention may be practiced without these specific details. In other instances, well-known methods, procedures and components have not been described in detail so as not to unnecessarily obscure aspects of the present invention. Furthermore, it should be understood that the possible permutations and combinations described herein are not meant to limit the invention. Specifically, variations that are not inconsistent may be mixed and matched as desired.

For the sake of convenience and simplicity, the terms “user,” “consumer,” and “registrant” may be used interchangeably herein, but are generally given their art-recognized meanings. In general, wherever one such term is used, it also encompasses the other terms. Similarly, for convenience and simplicity, the terms “party” and “entity,” and separately, the terms “individual” and “person,” and the terms “information” and “data,” are, in general, interchangeable and may be used interchangeably herein, but are generally given their art-recognized meanings, and wherever one such term is used, it also encompasses the other terms. For convenience and simplicity, the term “animal” as used herein refers to a non-human animal, and the term “living product” may refer to a living plant product and/or a living animal product, as those terms are defined and/or described herein. In addition, the terms “part,” “portion,” and “section” may be used interchangeably herein, but these terms are also generally given their art-recognized meanings. Also, unless indicated otherwise from the context of its use herein, the terms “known,” “fixed,” “given,” “certain” and “predetermined” generally refer to a value, quantity, parameter, constraint, condition, state, process, procedure, method, practice, or combination thereof that is, in theory, variable, but is typically set in advance and not varied thereafter when in use.

The present invention establishes a foundational identity layer for each individual plant, plant strain (for an asexually reproduced plant), individual animal and/or animal species, and/or living plant and/or animal product that moves through various stages in the supply chain, commercial channels and/or transportation system. By leveraging genetic information and blockchain technology, authenticity information stored in a secured data exchange (which may further include applicable regulations, safety information, logistics information, etc.) can be securely captured and validated through the entire chain from the origin to the end user, optionally using a smart phone app. In case of a specific incident (e.g., contamination with and/or an outbreak of a food-borne pathogen), authorities can retrace the validation access steps quickly to isolate and recall any contaminated products.

The present invention can further assist verification of authentic plants, animals, and living plant and animal products (e.g., plant seeds, cuttings, and fresh fruit and vegetable food products; stem cells, embryos, organs, and other living animal products) and help to identify counterfeit products and imposters, throughout commercial activity chains by retesting the genetic information at any point. Each test and retest may be captured in the blockchain and thus ensure transparency and security of each plant or animal tested or retested.

One goal of the present invention is to facilitate preparedness in and/or among the general public regarding emergency identification during a food crisis, and at the same time, directly support enforcement efforts, making the process of identifying certain plants, animals, and living plant and animal products in commercial activities faster, easier, and/or less expensive. Other goals of the present invention include enabling fast and facile identification of certain plants and/or animals and/or ownership and traceability of digital assets associated therewith (such as identification and other commercially useful information in electronic form) in a safe and secure manner. The present invention aims to decrease pressure, stress and/or reliance on limited public and private resources, especially during a significant event (such as large-scale contamination of a crop or an outbreak of an animal-borne disease).

The same principles used to identify humans using DNA analysis can apply to identification of non-human animals, such as pets (e.g., dogs, cats, birds, etc.) and livestock (e.g., cattle, horses, sheep, etc.). The present invention also provides an advanced method of identifying a plant, animal, or living plant or animal product, saving time and the commercial value of the plants, animals and/or products.

Identity is an important part of society, but it is even more critical during exigent circumstances. In some aspects of the present invention, users and/or owners own and/or manage access to and authorization of release of the identification information when it is needed. The present system and method can completely avoid invasive means of pet and livestock identification and can reliably authenticate asexually reproduced plants and living plant/animal products.

### Exemplary Methods

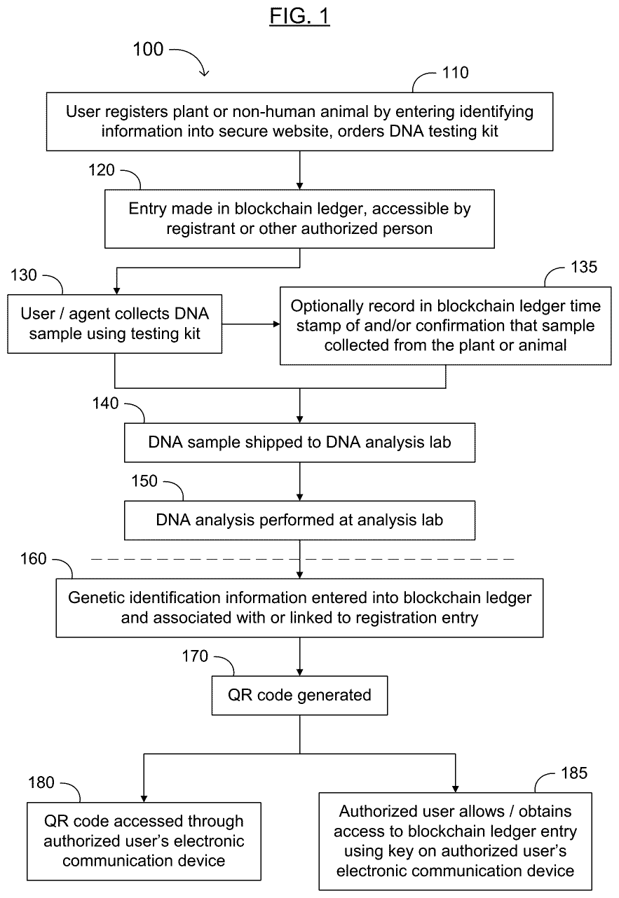

The present method may be divided into two processes: a testing/analysis process, and a digitalization/identification management process. An exemplary process for the present method is shown in the flow chart 100 in FIG. 1.

In a first step, a user may register a plant, non-human animal, or living plant or animal product with an administrator of the blockchain registry at 110 by entering identification information on a secure website. The user may be the owner of the plant, non-human animal, or living plant/animal product, a third party registering the plant, non-human animal or living plant/animal product for the owner, an employee or representative of a business or non-profit entity seeking to register the plant, non-human animal or living plant/animal product, or another person to whom authority has been granted by the owner. The user may register other plants or animals (e.g., other plants or animals owned by the same organization, such as a farm, a university, a government agency, a corporation, etc.) or other living plant/animal products who give the user authority to register the plants, animals or products on their behalf. For example, the animals may be pets, such as dogs, cats, birds, reptiles (e.g., snakes, iguanas, turtles, etc.), rabbits, etc., or livestock, such as cattle (bulls, cows, calves, etc.), horses, bison, poultry (e.g., chickens, geese, ducks, etc.), sheep, pigs, goats, etc. The plants may be sexually or asexually reproduced, but for purposes of verifying or authenticating the source or lineage of the plant, the present method and system are particularly appropriate for registering asexually reproduced plants. Living plant products may include seeds, cuttings, fresh fruit, fresh vegetables, etc. Living animal products may include stem cells, living tissues and organs, animal cell lines (natural or genetically engineered), embryos, etc.

The identification information entered into the secure website may include the species and (when applicable) breed or variety of the plant or animal being registered; the name(s) of the user, the owner(s), and if applicable, the animal being registered; birth information (e.g., date and/or place of birth) of the animal; reproduction information (e.g., date and/or place of reproduction) of the plant; production date and optionally certain production (e.g., harvest) information of the plant or animal product; biometric information (e.g., height, weight, color[s]); ownership information, such as home and/or mailing address, email address, phone number, and for organizations, jurisdiction of registration or incorporation of the owner; “multiple birth” status (i.e., the registrant is one of a multiple birth group, such as a litter), or a combination thereof. Optionally, the user may enter certain health or genetic information (e.g., known genetic modifications, such as a suppressed gene or tolerance to a particular drug or biocidal agent; exposure to a particular disease, etc.). In addition, the user may upload a photo of the plant, animal or living product to be registered. In some embodiments, the user may submit a relatively large number of entries using a Comma Separated Values (.csv) file for a batch registration of a crop, herd or harvest/production in a single submission. For example, the .cvs file may contain 5-100 individual animals (e.g., horses) to be registered.

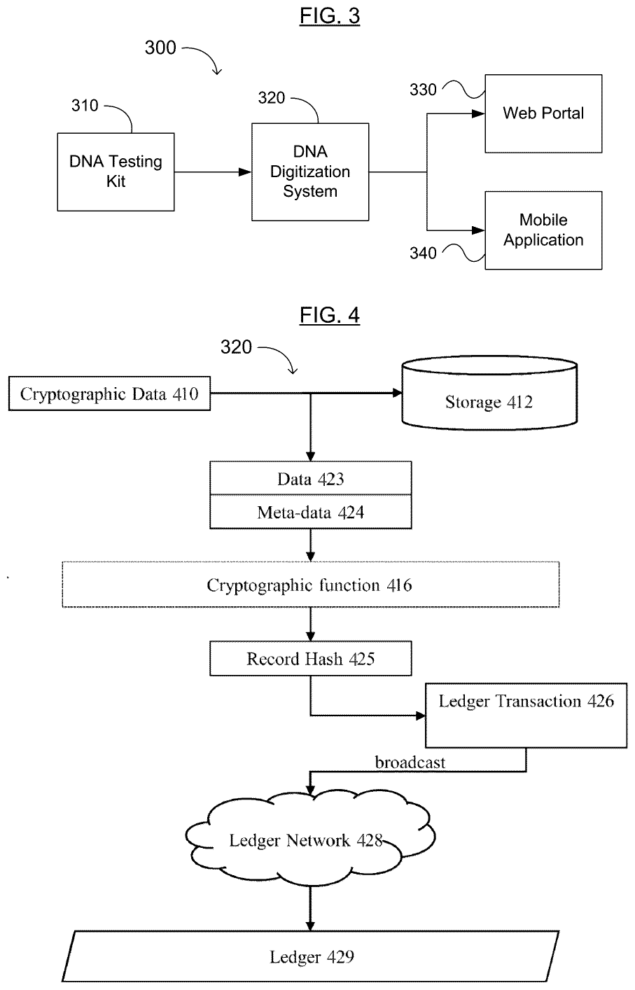

The user may complete registration by ordering a DNA testing kit at 110. The database/registry administrator generally confirms that no entry exists (e.g., in a blockchain register, other genetic identity database, etc.) for the plant, animal or living product to be registered prior to entry into the blockchain ledger at 120. If necessary or desired, the database/registry administrator resolves any potential duplication of genetic or other identification information to prevent any plant, animal or living product from having more than one genetic identity. The database/registry administrator may also charge a fee for the test kit and for shipping, and may collect any applicable tax. An entry is then made in a blockchain ledger at 120. The entry into the blockchain ledger is explained in greater detail with regard to FIGS. 3-4. The ledger entry is accessible to the user and the owner (if different from the user), as well as to those authorized to access the ledger (e.g., on behalf of the owner).

Alternatively, the user can simply enter identification information without ordering the DNA testing kit, if the registrant's non-DNA identification information is to be maintained in the database/registry without corresponding genetic identification information, but the benefits of access to the encrypted genetic identification information are lost. However, there may still be some benefits to storing the plant's, animal's or living product's non-DNA-based identification information in a blockchain registry.

The DNA testing kit is then shipped to the user or the designated custodian or authorized agent (e.g., caretaker or veterinarian). When the kit is received, a DNA sample is collected at 130 from the plant, animal or living product in accordance with the instructions in the kit. Optionally, an entry is recorded in the blockchain ledger at 135 with a time stamp for the sample collection and/or a confirmation that the sample was collected from the plant, animal or living product that was registered at 110. For example, the confirmation at 135 may comprise a certification or other written statement from the user, custodian or authorized agent confirming that the DNA sample was collected, or alternatively, from a third party confirming that they have the authority to collect the DNA sample(s). The third party may be registering (or collecting a sample from) a group or other plurality of plants or animals. In an alternative embodiment, limited identification information (e.g., owner name, email address, plant/animal species and breed, date of birth, and optionally other basic identification information sufficient to create a unique identifier for the plant/animal/product) is collected at 110, and the remainder of the identification information is collected at 130.

The sample is then shipped to the analysis lab at 140 for analysis. The user may include a user-generated PIN for maintaining confidentiality of the DNA test. Typically, the test kit contains a form and instructions for submitting the PIN with the kit. Alternatively, a shipping or delivery service can pick up the sample and deliver it to the lab. Typically, the test kit will include a pre-addressed, postage-paid envelope or container for shipping the sample to the lab. In a further option, an entry is recorded in the blockchain ledger with a time or date stamp for shipment of the sample to the analysis lab. For example, the entry can be made by the user or third party (e.g., using a camera on the user's or third party's communication device to generate one or more documentation photographs, such as of the bar code on the kit or the sample holder, the user or third party holding the sample ready to ship, etc.). Such photographic entries may be useful to validate the identity of the user or third party, at least in part.

At 150, the analysis lab conducts DNA testing. Kits and equipment for performing DNA testing are widely available. DNA testing for animals is primarily for individualization, parentage, ancestry, presence of genetic diseases, and color/pattern identification. In some embodiments, DNA testing involves comparing the results to known genetic markers, which in some cases, are known to be associated with a specific species or breed. The testing/analysis method may include short tandem repeat (STR) analysis, whole genomic and/or next generation DNA sequencing, mitochondrial DNA (mtDNA) sequencing, and analysis of single-nucleotide polymorphisms (SNPs), among other techniques. Although STR profiling is a well-known and reliable process for identifying individual plants or animals, in some cases (e.g., determining and/or authenticating horse parentage), SNP-based massively parallel sequencing (MPS) and/or whole genomic and/or next generation DNA sequencing is a well-accepted procedure.

DNA test/analysis kits and equipment is commercially available. For example, STR test kits are available from Thermo Fisher Scientific Corporation under the Applied Biosystems brand (Waltham, MA), Promega Corporation (Madison, WI), Qiagen (Germantown, MD) and others, and the application(s) thereof to confirming and/or authenticating identifications of individuals is disclosed at length in U.S. patent application Ser. No. 17/555,968, filed on Dec. 20, 2021, the relevant portions of which are incorporated herein by reference. Equipment such as genetic analyzers are available from Thermo Fisher Scientific and others. SNP testing (e.g., genotyping) can be performed using large-scale microarray technology (see, e.g., Nishigaki et al., “Extensive Screening System Using Suspension Array Technology to Detect Mitochondrial DNA Point Mutations,” Mitochondrion, 10 [2010], pp. 300-308, and/or Keating et al., “First All-in-One Diagnostic Tool for DNA Intelligence: Genome-Wide Inference of Biogeographic Ancestry, Appearance, Relatedness, and Sex with the Identitas v1 Forensic Chip,” Int. J. Leg. Med., 127, pp. 559-572, the relevant portions of which are incorporated herein by reference), TaqMan technology on a quantitative PCR (qPCR) instrument (see, e.g., Kidd et al., “Developing a SNP Panel for Forensic Identification of Individuals,” Forensic Sci. Int., 164 [2006], pp. 20-32, the relevant portions of which are incorporated herein by reference), or detection through capillary electrophoresis (CE) (see, e.g., Wang et al., “Expansion of a SNAPshot Assay to a 55-SNP Multiplex: Assay Enhancements, Validation, and Power of Forensic Science,” Electrophoresis, 37, pp. 1310-1317, the relevant portions of which are incorporated herein by reference). Commonly-used, commercially available MPS technologies include the Illumina Solexa sequencing-by-synthesis technology (see, e.g., Bentley et al., “Accurate whole human genome sequencing using reversible terminator chemistry,” Nature, 2008, 456 (7218), pp. 53-59, the relevant portions of which are incorporated herein by reference), Roche 454 pyrosequencing (see, e.g., Margulies et al., “Genome sequencing in microfabricated high-density picolitre reactors,” Nature, 2005, 437 (7057), pp. 376-380, the relevant portions of which are incorporated herein by reference), sequencing by oligonucleotide ligation and detection (SOLID) DNA sequencing technology available from Applied Biosystems/Thermo Fisher Scientific Corporation, MinION sequencing technology available from Oxford Nanopore Technologies, and PacBio single molecular real-time (SMRT) sequencing technology available from CD Genomics (Shirley, NY). Pacific Biosciences SMRT sequencing detects sequences using nucleotide-specific fluorescent dyes that are detected in real-time.

Such commercially available kits, which typically provide premixed primers and a standard master mixture containing the polymerase(s), enzyme buffers, and any dNTPs useful for amplifying the DNA, simplify generation of DNA profiles and provide results on a uniform set of core loci to make it possible to share genetic identification information and compare it with similar genetic identification information obtained from different samples. In fact, commercial kits are preferred in most analytical laboratories over in-house assays, even though the kits may be more expensive. Commercially-available kits help simplify and standardize procedures, and remove the burden of PCR component quality control from the analysis lab. In addition, such kits may supply allelic ladders or other known standards containing common alleles or DNA sequences of known content and length that have been previously characterized for DNA sequencing. These allelic ladders and known standards are used to calibrate amplification products and/or product sizes for genotyping purposes.

For example, the process for STR testing includes sample collection, DNA extraction, DNA quantitation, PCR amplification of multiple STR loci, STR allele separation and sizing, STR typing and profile interpretation, and a report of the statistical significance of a match (if observed). Following PCR amplification, the overall length of the STR amplicon is measured to determine the number of repeats present in each allele found in the DNA profile. This length measurement is made via a sized-based separation using gel electrophoresis or capillary electrophoresis (CE). Each STR amplicon may be fluorescently labeled during PCR when either the forward or reverse locus-specific primer contains a fluorescent dye. By recording the dye color and migration time of each DNA fragment relative to an internal size standard, the size for each STR allele may be determined following its separation from other STR alleles. Commonly-used instruments for STR allele separation and sizing include the ABI PRISM 3100 and ABI PRISM 3500 genetic analyzers (available from Thermo Fisher Scientific Corporation under the Applied Biosystems brand). The result of the STR test is series or plurality of graphs or plots of the size of the repeated DNA segments at a number of predetermined loci, as determined by gel electrophoresis or capillary electrophoresis. Usually, the number of loci is from 5 to 25 (the greater the number of loci, the greater the confidence in the results), and the number of graphs or plots is based on the number of electrophoresis separations run in the test/analysis.

Plant DNA testing may involve genotyping, variety identification, and hybrid purity testing. Plant DNA can be analyzed using various methods, including genotyping by sequencing (GBS, which also uses restriction enzymes to reduce genome complexity and genotype multiple DNA samples; also see U.S. Pat. No. 9,951,384, the relevant portions of which are incorporated herein by reference), next-generation sequencing (NGS), polymerase chain reaction (PCR)-based sequencing, SNPs or simple sequence repeat (SSR) DNA markers, and 3^{rd} generation sequencing, all of which are commercially available. For example, GBS-based sequencing/analysis services are available from Freedom Markers, Ames, IA; NGS-based sequencing/analysis services are available from Creative Biogene, Shirley, NY and Daicel Arbor Biosciences, Ann Arbor, MI, and test kits and equipment are available from Twist Biosciences, South San Francisco, CA; and 3^{rd} generation sequencing services are available from Helicos BioSciences/SeqLL Inc. (Billerica, MA), Twist Biosciences, Pacific Biosciences (Menlo Park, CA), and others.

Plant DNA analysis is used in agricultural research for crop trait development, seed quality control, determination of disease resistance, population genetics, as well as in forensic botany. 3^{rd} generation sequencing (TGS) has certain advantages over NGS in that the reads are longer, which can fill in gaps previously missed by NGS techniques. MinION TGS, created by Oxford Nanopore Technologies (Oxford, UK), enables sequencing in a portable format (e.g., for use in remote locations and/or challenging environmental conditions). Benefits of TGS include reductions in single-base mistakes (relative to NGS), more accurate identification of mutations, reductions or avoidance of false positive results, avoidance or elimination of PCR, long readings, and relatively even coverage.

Referring back to FIG. 1, a digitalized format of the plant's, animal's or living product's DNA analysis result (i.e., the genetic identification information) is reported directly to authorized individuals (e.g., the owner, any authorized agents, etc.) and entered into the blockchain ledger at 160. At the same time, the genetic identification information and/or the blockchain ledger entry are associated with the registration entry in the blockchain ledger. In some embodiments, the analysis lab encrypts the DNA analysis result before uploading (e.g., to the database/registry administrator or directly to the blockchain ledger), optionally using the PIN included with the collection kit, so that the genetic identification information is encrypted before it is entered into the blockchain ledger. In one variation of these embodiments, only the owner or other authorized user can decrypt the genetic identification information. This variation protects the data from access by third parties and unauthorized entities, who may do so innocently or with malicious intent. Once the owner or authorized user receives the DNA analysis result, they can decrypt it using a decryption key (which may be a public key generated by the registrant, or the encryption key [or a complement thereof] programmed into a secure application provided by the database/registry administrator). In some examples, the DNA analysis result may be a further encrypted using a combination of the plant's, animal's or living product's identifying information and/or DNA sequence before it is entered into the blockchain ledger.

At 170, the digital DNA analysis result (which may be previously encrypted) is embedded as a unique machine-readable icon or other symbol, such as a QR Code, a bar code, etc. For example, the machine-readable symbol may be a digital representation of the digital DNA analysis result and may comprise locus, allele and STR copy number information, converted to a digital format. In various embodiments, the digital format may comprise p characters, where p is an integer of (2^{q}+2^{r}), q is an integer of 5 or more, and r is 0 or an integer of one or more. In one example, p is 196. In a further embodiment, the digital format may be condensed or compressed using a conventional algorithm (e.g., to a smaller number of characters). The options for obtaining and testing the sample and for recording, reporting, displaying and otherwise using the test results are virtually unlimited.

For example, the owner or other authorized user can share the plant's, animal's or living product's genetic identification information with pre-approved individuals or entities, or present such information to a governmental or other authority in emergency situation (for example, by providing a decryption key to such individuals, entities or authorities). For example, genetic identification information can be presented by showing the QR code or similar information displayed on the electronic communication device of the owner, other authorized user or pre-approved individual or entity at 180. When the genetic identification information in the blockchain ledger is encrypted, the QR code or similar information is presented after decryption. Alternatively or additionally, genetic identification information can be shared with pre-approved individuals or entities by sharing the access information and decryption key with the pre-approved individuals or entities at 185.

The genetic identification information may be managed in different ways. In one example, a private permission identification information management system is implementable as a blockchain network, and can accept and retain identification information as well as genetic identification information. Identification information (which can be entered by the registrant or authorized user at the time of registration at 110 or at the time of collecting the DNA sample at 130) can include names of the owner and of the plant or animal, an age or date of birth/reproduction, place of birth/reproduction, instructions on how to care for (and/or alternatively dispose of) the plant, animal or living product in case of an emergency, damage or injury, etc. Thus, the genetic identification in the present invention can be used to authenticate the plant, animal, or living product and/or the plant's, animal's, or living product's identification information. Other documents, such as copies of the plant's or animal's birth/reproduction records, vaccination information, auction results, lineage, breeding information, etc., or the living product's production or harvest information (e.g., date of production or harvest, name and location of the production or harvest facility, lot no., storage conditions, etc.), may also be stored (e.g., as one or more additional blockchain entries). The stored and/or associated documents may also be authenticated using the plant's, animal's, or living product's genetic identification.

With blockchain-based transactions, contracts related to the plant, animal or living product can be embedded in digital code and stored in transparent, shared databases, where they are protected from deletion, tampering, and revision. As a result, substantially every agreement, medical treatment or procedure, exchange of consideration (e.g., money), etc., related to the plant, animal or living product can have a digital record (and, where relevant, a signature) that can be identified, validated, stored, and shared. One aspect of the present invention keys the validation of the digital code to a unique genetic DNA record that may not be altered or superseded. Intermediaries such as lawyers, brokers, bankers, etc., may not be necessary for such activities. Individuals, organizations, machines, and algorithms can therefore freely interact with one another and conduct transactions with little friction. These are among the many advantages of blockchain.

### Exemplary Systems for Use of Plant, Animal and Living Plant or Animal Product Genetic Information

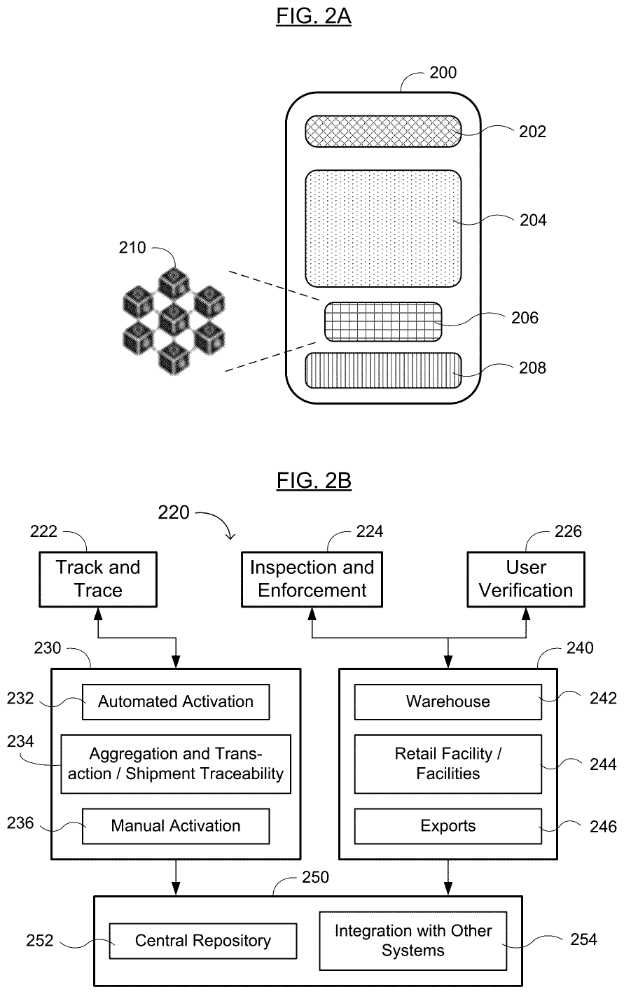

FIG. 2A shows an exemplary genetic ID display or card 200 for a plant, animal or living plant or animal product. The display may be on an electronic communication device, such as a smart phone or a tablet computer. The card may be similar to a conventional credit card or organizational ID card, and may be electronically readable (e.g., using an RF-based or near-field reader, such as a smart phone or other hand-held electronic reading device).

The display or card 200 may comprise a plurality of regions therein or thereon, such as a logo or vendor identification region 202, a visual identification region 204, a code region 206, and a linking region 208. The logo or vendor identification region 202 may include a picture or drawing of the genetic ID information analysis and/or storage service provider, the owner or owner's enterprise/organization, etc., and may include a hyperlink to a website of the service provider or owner. The visual identification region 204 may include a picture or drawing of the specific plant, animal or living product, the species of the plant or animal, or a generic picture of the living plant or animal product associated with the genetic ID. The code region 206 contains the code corresponding to the genetic ID of the specific plant, animal, or living plant or animal product. The code displayed in the code region 206 may be a QR code, a bar code, or other conventional optically or magnetically scannable code. By scanning the code in the code region 206, one may validate the plant, animal or living product at each step in the transportation or import/export/regulatory channel, and can be used before, during and after a commercial transaction or transport to trace the authenticated item. The linking region 208 may contain a different hyperlink to a website (optionally a secure website) or other information storage location from which the identification information (and optionally other information) stored in the blockchain can be retrieved and displayed on the electronic communication device or card reader.

FIG. 2B shows exemplary systems and functions in a digital track-and-trace system and method 220 in accordance with the present invention. At each point in the stream of commerce (e.g., at transportation points, such as pick-up and delivery, loading and unloading, storage in or transfer at a warehouse, etc., and at commercial events, such as inspection, contract formation/sale, payment, recordation of title, etc.), the registered plant, animal or living product can be tacked and/or verified, and an entry made in the associated blockchain.

In various embodiments, tracking and/or tracing the plant, animal or living product may be desired after the plant, animal or living product is purchased, sold or shipped (e.g., transported to another location). For example, after the plant's, animal's, or living product's genetic ID is created and identification information is stored in a blockchain, the owner or authorized user may track and/or trace the plant, animal or living product at 222 using one of more tracking/tracing services or mechanisms 230. In one example, the owner or authorized user may track and/or trace the plant or animal using automated activation at 232. Automated activation can be authorized by the owner or authorized user during or after registration. Alternatively, the owner or authorized user may track and/or trace the plant, animal or living product using manual activation at 236. Manual activation may comprise a conventional authentication protocol, such as those commonly used in online commercial transactions (e.g., accessing a secure website, making an online purchase, etc.). In another example involving multiple plants, animals or living products, the genetic IDs may be conventionally aggregated at 234 to facilitate traceability of all of the plants, animals or living products in a particular transaction or shipment. Such aggregation may further include serializing the individual plant, animal or product genetic IDs. Following the initial aggregation for a particular transaction or shipment, any subsequent rework or re-aggregation (e.g., as a result of a subsequent transaction or shipment involving the plants, animals or living products in the original transaction or shipment) can be similarly traced and/or tracked.

After the plants, animals or living products enter the stream of commerce at 240, they may be delivered to a warehouse at 242, one or more retail facilities at 244, or one or more export facilities or channels at 246. The owner or authorized user may verify the location and/or stage in the commerce stream of the plant(s), animal(s) or living product(s) at 226. Additionally, an officer of an inspection or enforcement agency or bureau may access the genetic ID and associated information in the blockchain for the plant(s), animal(s) or living product(s) at 224, as needed. This is particularly useful for import/export and backend traceability of the plants, animals and living products, and can limit the extent to which the plants, animals or living products must be isolated and/or destroyed as a result of a disease outbreak or contamination issue. Authorization for the officer to access the genetic ID and associated information may be given automatically at 232 (e.g., by the owner or user, or by the service provider if the service provider is required to do so) or manually at 236 (e.g., upon request by the officer).

The digital track-and-trace system and method 220 further includes a storage and integration system 250. For example, the tracking/tracing services or mechanisms 230 and the events occurring in the stream of commerce 240 may be stored in a central repository at 252 (e.g., in the blockchain associated with the plant[s], animal[s], or living product[s]). In addition, the genetic ID and associated information in the blockchain for the plant(s), animal(s) or living product(s) may be accessible to/by other systems, such as regulatory and governmental databases, other computer and/or electronic storage systems of the owner and/or genetic ID service provider, etc., at 254. In some embodiments, integration with other system(s) may comprise two-way data communications with the other system(s), providing a validation key that may be embedded real-time in the QR code, barcode, text, or other presentation or representation of the identification data. This provides for 3^{rd} party authorization and/or authentication at that time. For example, if a user registers a dog for a dog show, the dog show authority may send a validation code to allow entry to the show ring for one or more events for which the user is authorized to attend or participate. The dog show staff can then scan a digital display code, such as on a smartphone, to screen entry to a secure event.

FIG. 3 shows a block diagram of a system and/or hardware 200 for implementing a method of obtaining and providing genetic identification information for plants, non-human animals, and living plant and animal products in accordance with embodiments of the present invention. The system and/or hardware 300 comprises a genetic material sampling kit (e.g., DNA testing kit 310), a genetic information digitization system 320, a web portal 330 and a mobile application 340.

The DNA testing/analysis kit 310 may comprise a sealable container configured to sealably contain a sample containing genetic material of the plant(s), non-human animal(s), or living plant or animal product(s), written instructions for (i) taking the sample, (ii) providing an optional PIN, and (iii) placing the sample in the sealable container, and a pre-addressed envelope or box for sending the sample in the sealable container to a genetic material analysis facility. The DNA testing kit 310 may comprise a sample-taking kit that is used commercially (e.g., in a veterinary office or farm, a testing service provider facility, a forensic lab, etc.). In various embodiments, the sealable container comprises a sealable plastic bag or a vial or tube with a cap or lid configured to seal an opening in the vial or tube. The DNA analysis kit may comprise (i) a gel electrophoresis cassette/tray and a gel or (ii) a capillary electrophoresis capillary, primers that may further include a fluorescent or luminescent label, and enzymes and reagents to process and/or amplify the DNA. The system 300 may further comprise a genetic analyzer (e.g., to analyze the genetic information supplied in the sealable container).

The system and/or hardware 300 may further include a first electronic communication device configured to enter personal information of the registrant to a secure website, a second electronic communication device configured to record the personal information and the genetic identity in a blockchain ledger, and a third electronic communication device configured to display a code corresponding to the genetic identity. Typically, the second electronic communication device is different from the first and third electronic communication devices, and the first and third electronic communication devices may be the same electronic communication device or different electronic communication devices.

The first electronic communication device may comprise a personal computer or a smart phone, which may be configured (e.g., with an app 340) to enter at least two of a name, contact information of the owner, an identification number issued by a governmental or other (e.g., industry-based) authority, and a photograph of the plant, animal or living plant or animal product as the identification information. The owner's contact information may include the owner's name, mailing address, email address, phone number, website address or uniform resource locator (URL), or a combination thereof. In some embodiments, the first electronic communication device may be further configured to enable the owner or authorized user to (i) register for a service comprising the DNA analysis and recordation of the identification information and genetic identity, and/or (ii) access and/or display a code corresponding to the genetic identity. The second electronic communication device may comprise a personal computer, a workstation, or a server, for example, and may be configured to offer (a) a service comprising the DNA analysis of the genetic material, recordation of the personal information and genetic identity, and/or creation of a code corresponding to the genetic identity of the plant, animal, or living product and/or (b) the genetic material sampling kit to the owner or authorized user. The second and/or third electronic communication devices may be further configured to authenticate an identity or personal information of the plant or animal using its genetic identity, and the third electronic communication device may be further configured to access the code from the blockchain ledger.

The genetic information digitization system 320 is largely conventional. An example of a genetic identification management system 320 is shown in FIG. 4, which includes a blockchain distributed ledger 429 that stores encoded data relating to the plant's, animal's, or living product's genetic identification. The ledger 429 may be an immutable distributed ledger, and the blockchain may include, for example, a public blockchain and/or a private blockchain. In some embodiments, the storage 412 may be the same as the ledger 429. The system 320 in FIG. 4 provides safety and integrity for multiple records and events within the system 320, all within the parameters of a single ledger transaction 426 on the ledger 429.

Each new record (or combination of records) of a transaction in the storage medium 412 generates a ledger transaction 426 into the ledger 429, which allows anyone to verify and validate the existence and accuracy of the data entry. One embodiment of verification includes analyzing the cryptographic data 410 in combination with a digital signature for the ledger transaction 426 that is provided to the ledger 429. Advantageously, anyone can validate the existence of the information in the ledger transaction 426 based on the cryptographic data 410 using the storage 412 and the ledger 429. As shown in FIG. 4, the cryptographic (e.g., identification) data 410 is stored in the storage 412, while also being divided into core data 423 and metadata 424. Metadata 424 is generally (but not always) not present within the cryptographic data 410, so core data 423 may be equal to or the same as the cryptographic data 410. Metadata 424 can be derived from external sources (not shown) and/or determined from other variables (e.g., timestamps). Both the core data 423 and the metadata 424 can be processed using the cryptographic function 416.

A record hash 425 is generated from the core data 423 and, when present, the metadata 424. The record hash 425 is distributed to the ledger transaction 426 as additional information. For a blockchain transaction, the record hash 425 may be written into an ‘OP_RETURN’ field of the ledger transaction 426. The ledger transaction 426 is broadcast over a ledger network 428. As soon as a new block (reflecting the transaction) is created on the ledger 429, the record(s) that the system 320 has placed within the ledger transaction 426 is/are secured inside the ledger 429 itself. In other words, when the ledger transaction 426 is in the block, it is difficult or impossible to change it or tamper with it, so it is difficult or impossible to change its history. Anyone in possession of the corresponding raw data can produce the cryptographic data 410, check its existence within the storage 412, and validate/verify information using the ledger 429.

Furthermore, in some embodiments, the storage medium 412 does not maintain data in its original or open form. In contrast, the raw data can be first processed through the cryptographic function 416 as shown in FIG. 4. This is advantageous in that hashed stored data cannot be reverse-engineered back to its original form, even if a hacker were to obtain access to the hashed data. In some embodiments, the genetic identification management system 320 can have at least one processor (e.g., in the registrant's electronic communication device) configured to perform cryptography primitives on identification information/data sets (e.g., the raw data and/or the cryptographic data 410).

Any input into the storage 412 as described herein may be followed by the generation of one or more ledger transactions 426 made in the ledger 429 as shown in FIG. 4, to provide a fully secured and trusted way of immutable data storage, validation and/or verification, and authentication. As used herein, the term “immutable data” refers to data that, once originated, never changes (e.g., names of parents or parent organism, date of birth/propagation, biological sex, multiple birth status, place of birth, etc.) or that that is difficult to manipulate, for example, even for a system administrator, after the data has been written to a blockchain.

Each individual user of the system 320, such as an owner or an authorized user, can be issued with a cryptographic secret key (such as a private key), which in some embodiments is relatively long. In some embodiments, the cryptographic secret key can comprise a Rivest-Shamir-Adleman (RSA) key, an elliptic curve cryptography (ECC) key, or the like. The known features of ECC enable this type of key to be split into a plurality of independent parts (factors). These factors can be of any nature, such as tokens, passwords, biometric data, pin-codes and the like, but are not limited to these examples.

In one common model for how blockchain works, there are five basic principles underlying blockchain technology. First, a distributed database is used. Each party on a blockchain has access to the entire database and its complete history. No single party controls the data or the information therein. Every party can verify the records of its transaction partners directly, without an intermediary.

Second, transactions and communications are conducted by peer-to-peer transmission. In other words, communication occurs directly between peers, instead of through a central node. Each node in a transaction or communication stores and forwards information to all other nodes in the transaction or communication.

Third, transactions on a blockchain are transparent, but the participants are not easily identified (i.e., there is pseudonymity). Every blockchain transaction and its associated value are visible to anyone with access to the system containing the blockchain. Each node, or user, on a blockchain has a unique 30-plus-character alphanumeric address that identifies the node/user. Users can choose to remain anonymous or provide proof of their identity to others, at the user's option. Transactions occur between blockchain addresses.

Fourth, records on a blockchain are irreversible. After a transaction is entered in the database and the accounts are updated, the records cannot be altered, because they are linked to every transaction record that came before them (hence the term “chain”). Various computational algorithms and approaches are deployed to ensure that the recording on the database is permanent, chronologically ordered, and available to all others on the network.

Fifth, blockchains use the computational logic available in the network/system that includes them. The digital nature of the blockchain ledger means that transactions recorded therein can be tied to computational logic, and in essence, programmed. As a result, users can set up algorithms and rules that automatically trigger transactions between nodes.

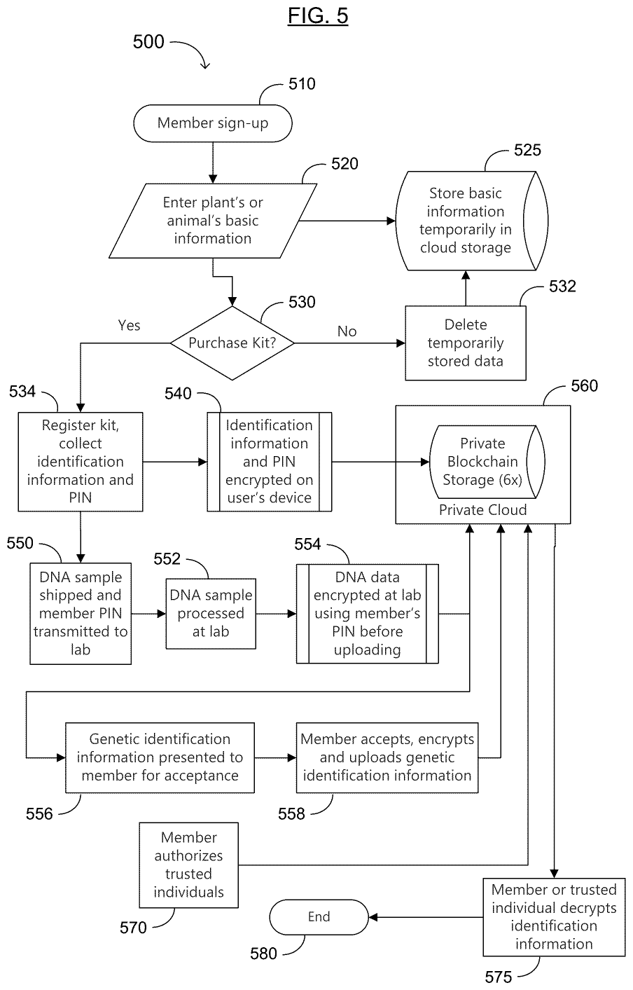

FIG. 5 shows a flow chart 500 for another exemplary method in accordance with embodiments of the present invention. The flow chart 500 is consistent with the flow chart 100 in FIG. 1, but may contain some variations and/or details that are not present in or discussed with regard to FIG. 1.

The method of FIG. 5 starts at 510 when a member (e.g., the owner or an authorized user) signs up, for example, on a website or using an app. Typically, signing up comprises entering an email address or other personal communication information (e.g., a mobile phone number, a social media handle or username, etc.) of the user, and optionally, requesting information about services related to the method.

At 520, the basic information of the plant, animal or living product is entered (e.g., into fields on a secure page of the website or into fields in the app). The basic information entered is generally a subset of the identification information for the flow chart 100 of FIG. 1, and may include the plant's or animal's name, the common name or genus, species and optionally breed or variety of the plant or animal, birth or propagation information, production or harvest information of the living plant or animal product, the owner's mailing and/or business address, and optionally the owner's or authorized user's phone number and email address and any additional organizational affiliation of the owner, plant, animal or living product. The basic information of the plant, animal or living product is then stored temporarily in a cloud data storage system at 522.

At 530, the user decides whether to purchase a kit (e.g., a DNA testing or sample collection kit) as described herein. If the user decides not to purchase the kit, the data temporarily stored in cloud storage is deleted at 532. However, when the user purchases the kit, the kit (e.g., its serial number or other unique identifier) is recorded on a blockchain register at 534, and the remainder of the plant's, animal's, or living product's identification information and a personal identification number (PIN) for the user or member are collected (e.g., by entry into fields on the website or app). The identification information may be selected from the identification information for the flow chart 100 of FIG. 1, other than and/or in addition to the basic information entered at 520. Optionally, the user also selects and enters a PIN, which may be n characters in length, where n is an integer of 6 or more (e.g., 6, 8, etc., optionally up to 12, 16, 20 or 24). The characters may be numbers, letters, or a combination thereof, optionally with one or more special characters, such as @, #, $, \hat{ }, &, *, a punctuation mark, etc. Also, after purchasing the kit, the user may be reclassified as a “member” or registrant.

At 540, the identification information and the member's or user's PIN are encrypted on the device into which the identification information and PIN were entered. Data entering the present genetic identification management system (including that entered at the user device) is encrypted using a local application on the local device (in this case, the user device) before uploading into the system. The algorithm(s) used to encrypt the identification information and PIN may comprise one or more conventional (e.g., industry-standard, current best practice, etc.) algorithms, such as Asymmetric Encryption (AE), Advanced Encryption Standard (AES) or Blowfish, or a public key cryptography algorithm such as RSA. The encryption key is generated using a symmetric encryption technique, where the originator (e.g., the user or member) creates a key and shares it with an entity (e.g., the DNA testing facility, the individual whose genetic identification is being entered and managed, etc.) that needs to upload data on their behalf. For example, when a member submits the plant or animal DNA sample to a lab or other testing facility along with their key at 550, the lab/testing facility uses the key to encrypt and upload the data at 554. The member (e.g., owner or authorized user) then uses their original key (e.g., PIN) to accept the data into an account associated with the member and optionally the plant, animal or living product at the genetic identification management service provider at 556 (i.e., after uploading at 554, but prior to storage in the private blockchain ledger at 560). After acceptance at 558 (described in more detail below), the encrypted data is stored in the private blockchain register at 560. In one embodiment, the member is allowed to use the PIN once and only once (e.g., given a one-time use of the PIN) to decrypt, and thus accept, the DNA testing data. On uploading to account and/or upon storage in the private blockchain data storage system at 560, the information undergoes further encryption utilizing one or more standard blockchain hashtag algorithms.

In greater detail, at 550, the user/member takes a DNA sample using the kit as described herein and ships the sample along with their PIN to the DNA testing facility (e.g., laboratory). Alternatively, the kit (sample) may be physically shipped to the DNA testing facility, and the PIN may be transmitted electronically (and optionally, securely) to the DNA testing facility. At 552, the DNA sample is processed at the lab as described herein to obtain a digitalized version of the plant's, animal's, or living product's genetic identification information (i.e., a code based on the graphs or plots of the plant's, animal's, or living product's DNA data, optionally at a number of predetermined loci). This digitalized DNA data is encrypted at the lab (e.g., using one or more AE, AES and/or RSA encryption algorithms) using the user/member's PIN as an encryption key at 554 before it is uploaded to the private blockchain register in a private cloud storage system at 560. For even greater security, on data acceptance (e.g., in the private blockchain register at 560), all data may be decrypted, then re-encrypted using one or more AE or RSA algorithms.

At or about the same time that the encrypted genetic identification information is uploaded to the private blockchain register, it is also presented to (e.g., transmitted to, made available through the secure website or app, etc.) the member at 556 for acceptance. The member may accept the genetic identification information at 558 by decrypting it (e.g., using the PIN as a decryption key), viewing and confirming the acceptability of the decrypted genetic identification information (e.g., by checking a box on the secure website or app, sending a message to the genetic identity information service provider or administrator, etc.), re-encrypting the decrypted genetic identification information (e.g., using one or more standard and optionally embedded encryption algorithms on the secure website or app), and uploading the encrypted genetic identification information to the private blockchain register at 560.

At 570, the member may share the encrypted identification information stored at 560 with trusted individuals, groups or entities, with public keys being generated on the member's device as needed (e.g., to be provided to the trusted individual, group or entity, for either a single use or for multiple or recurring uses). In some embodiments, the user/member authorizes a particular device to receive the key (e.g., based on a random sequence of the user's/member's identification information/DNA). The trusted individual, group or entity then uses the authorized device to enter a secure website (which may be the same as or different from that used by the user to register the member) or application programmed to enable access to (e.g., decrypt) the encrypted data by the trusted individual, group or entity.

At 570, the assignment and distribution of public keys (as selected by the member) to the trusted individuals, groups or entities is managed, for example using the secure website (which may be the same as or different from that used by the user to register the member) or application. The information by which a member or user accesses the system (typically a combination of email, password and system-determined identification information multi-factor authentication data from the registrant's identification information entries) is used to validate entry into the secure website or application and authorize generation of public keys. In one embodiment, the decryption key is generated from a unique combination of the user's/member's identification information and a fragment or sequence of the plant's, animal's or living product's DNA (e.g., an n-character sequence generated using a random sequence of the plant's, animal's or living product's DNA) as a seed. One benefit of the DNA-as-a-seed approach is that the plant's, animal's, or living product's DNA cannot be changed, unlike a name or identification number on a tag. This decryption key may be similar to an authorization code transmitted by the owner or administrator of a secure website to an authorized individual for access to the secure website. The random sequence may be 6 or more bases long (e.g., 8 or more, 10 or more, 12 or more, etc.), up to about 100 bases long. Although there is no technical upper limit to the number of bases in the random sequence, typically no more than 50-60 bases are necessary or desired in the sequence.

The member or the trusted individual, group or entity then decrypts and accesses the encrypted identification information (e.g., using the secure website or application and the public key) at 575. The decryption of identification information and DNA may occur at later date, as long as trust is not revoked by the member (or other entity with such revocation privileges, such as the owner), and may be utilized to assist in establishing the identity of the plant or animal. In general, the key for decryption resides with the authority controlling the genetic identification information (e.g., the individual member when the individual member registers themselves, a corporation or governmental agency, bureau or other entity when the corporation or government entity registers the member, etc.). The method 500 may then end at 580, or return to 570 when the member wishes to generate and/or distribute another public key and authorize another trusted individual, group or entity.

Returning to 560, the encrypted identification information may be segmented in the private blockchain storage system so that only specific subsets of the data (e.g., name, photograph, genus, species and breed or variety, parentage, a contract and/or purchase agreement, contact information, shareable and/or confidential documents such as intellectual property documents or trade secret information such as price or weight, security information such as permissions to access certain systems, etc.) are available to designated ones of the trusted individuals, groups or entities. More sensitive data, such as the plant's, animal's, or living product's genetic identification information, may be subject to additional protections that include converting an industry standard DNA information exchange format to a proprietary format which represents the plant's, animal's, or living product's raw or native DNA (e.g., in a format not generally recognizable by others, such as an n-digit-long numeric string that encodes the DNA information by digit and/or position in the string), optionally compressing the converted information, and then applying one or more industry-standard encryption algorithms to the compressed or uncompressed converted information. In some embodiments, the multi-level protection possible in the method 500 allows for generation of an individualized, unique identification number that may be used in genetic identification information management applications, the QR code for the member, or other identification methodology that does not expose the plant's, animal's, or living product's actual DNA information.

Further embodiments of the present invention relate to a unique architecture for managing self-sovereign identity (SSI) information (e.g., when the owner/registrant is an individual) and identification information (e.g., when the owner/registrant is an organization). As described above with regard to the method/flow 500 in FIG. 5, identification information data and transactional data (e.g., relating to registration) is encrypted and stored in the blocks of a private blockchain data repository. In this architecture, identification information may (and typically does) include DNA information. Endpoint encryption (e.g., on the user's electronic communications device) may be based on a system-generated encryption key, generated from a unique combination of the member's identification information and a fragment or sequence of the plant's or animal's DNA (e.g., an n-character sequence generated as described herein), using a random sequence of the plant's or animal's DNA as a seed. This system-generated encryption key may function as a private key for the member.

As described above, the member generates a private/public security key when creating an information profile. The private/public security key is used for encryption and decryption of the data in the system after the initial data upload. In some embodiments, only the member has access to the private key, and may decrypt data stored in the blockchain ledger using the private key (e.g., through system login validation). In such embodiments, the genetic identification information management service provider does not process, retain, or have access to any unencrypted data. In other or further embodiments, the member may share the public keys with one or more trusted persons or organizations through the genetic identification information management system. The public key provides the trusted person(s) or organization(s) with the ability to decrypt the plant's, animal's or living product's genetic identification data and/or other identification information in case of need.

Several levels of decryption may be available in the genetic identification information management system to enable separate access to common or basic identification information, secure documents, and/or the genetic identification information. For example, all such information may be encrypted using a conventional encryption algorithm, thus providing a first level of decryption. Certain information (e.g., secure documents and/or the genetic identification information) may be compressed (e.g., prior to encryption), thus providing a second level of decryption to enable separate access to such information. Furthermore, the plant's or animal's genetic identification information may first be converted to a digital (e.g., p-character) format, as described herein, before compression and/or encryption, thus providing a third level of decryption to enable separate access to the plant's or animal's genetic identification information.

Using the decryption algorithms and segments of the encrypted DNA sequence (i.e., the “seed), real-time validation challenge tokens may be generated. The challenge tokens may comprise a code such as a QR code, a bar code, or an authorization code. These tokens may be exchanged programmatically, scanned, typed, or communicated verbally to validate or authenticate the plant's, animal's or living product's identity based on the corresponding DNA sequence data. This validation may be a part of a multi-factor identification solution, as described herein.

In this manner, the encrypted DNA information typically cannot be matched to familial DNA (with the possible exceptions of asexually reproduced plants and cloned animals), which may be accessible to others or in the public domain. Accordingly, it is not possible to obtain a plant's or animal's DNA information or genetic identity from information that is available to others.

The present genetic identification information management system may also allow members who do not provide a DNA sample to replace the DNA sequence encryption/decryption seed with a numeric string representation from a photograph or other digital representation of a unique identifier.

In some cases, the genetic identity will be requested by a third-party authority such as governmental agency, a corporate entity, or an industry group. The genetic identification information management system or architecture may segregate data repositories (e.g., blockchain registers) so that the owner/member may retain SSI over data that the owner/member designates as SSI (e.g., stored in one repository or an SSI segment of a repository), and the third-party authority may access certain information to be shared (e.g., that is stored in a separate repository or in a “sharable” segment of the one repository).

The authorized device data and genetic identification code (e.g., QR code or bar code, e.g., in code region 206, FIG. 2A) may also be modified by the authority on a real-time basis in order to provide a validation key that certifies the currency (e.g., “most recent” status of the data) and/or validity of the genetic identification code as of the time of use. Implementations include placing or embedding the genetic identification code or other information to be presented using an electronic communication device, or on physical items such as ID badges, wallet cards, RFID tags or chips, ear tags, tattoos, laser-engraved tags, or wearables. Such physical items may also include an authority validation code placed thereon or embedded therein.

The genetic identification information management system or architecture may include an application programming interface (API) that enables genetic identification and other information in the management system or architecture and available to the authority to be exchanged, in a two-way manner, with other established information systems of, or controlled by, the authority. The API respects (e.g., cannot bypass) the encryption/decryption algorithms, and allows access to and exchange of only that information allowed by the individual member/registrant to be shared with the authority. In some embodiments of the two-way API, the authority may have the ability to push an encrypted security key to the genetic identification information management system or architecture. This allows the authority to include a software key for authority-controlled data, such as the individual's genetic identification code (e.g., QR code) or the underlying genetic identification data, stored documents, or other data that the individual cannot access. This would be utilized in the event of suspected compromise of the login validation, such as lost/stolen device/ID, suspected duress, or separation from the authority.

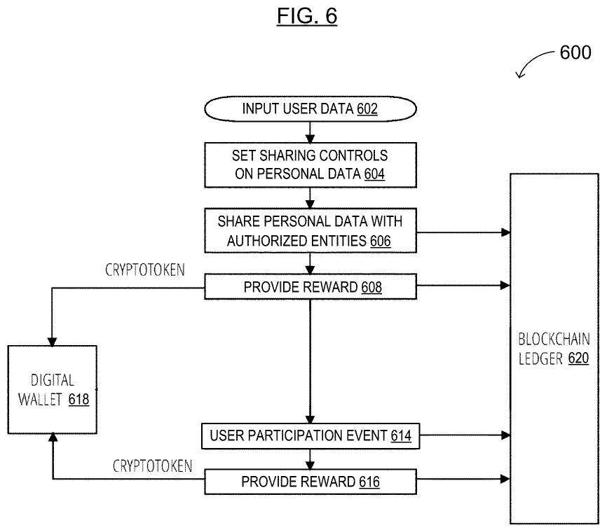

To encourage participation in a public permission genetic identification information blockchain network, a reward and data sharing process may be used. FIG. 6 shows a flow chart 600 for an exemplary reward and data sharing process, which begins by inputting user data (and, typically, identification information of the plant, animal or living plant or animal product) at 602, and setting sharing controls on the personal data at 604. These actions can also take place during user registration 110 (FIG. 1).

The reward and data sharing process 600 may share some or all of the user data and, if or when required, the identification information with authorized entities at 606, such as governmental authorities, veterinary care service providers, security service providers, insurance companies, etc. The specific parties or types of parties with whom the user data and identification information may be shared is also defined at 606. The authorizations and/or permissions for sharing the user data and identification information with other parties and/or entities are written into the blockchain ledger at 620. When the user/registrant provides at least some personal information of the user or owner, the process 600 provides a reward to the user/registrant at 608 via a digital wallet 618. The reward may be commensurate with the amount and/or type of information shared and/or the number and/or types of third parties given access to the information.

At various times, the process 600 may initiate a user participation event such as a survey questionnaire or a purchasing opportunity at 614. When the user/registrant participates in the user participation event, the process 600 provides another reward at 616. The reward may be commensurate with the amount of money spent and/or the total number of user participation events in which the user has participated.

The rewards may be deposited in the digital wallet 618 as cryptotokens, other electronic currency, or as discounts on products or services offered by participating sellers or providers. The transactions at 606, 608, 614 and 616 are recorded on the blockchain ledger 620.

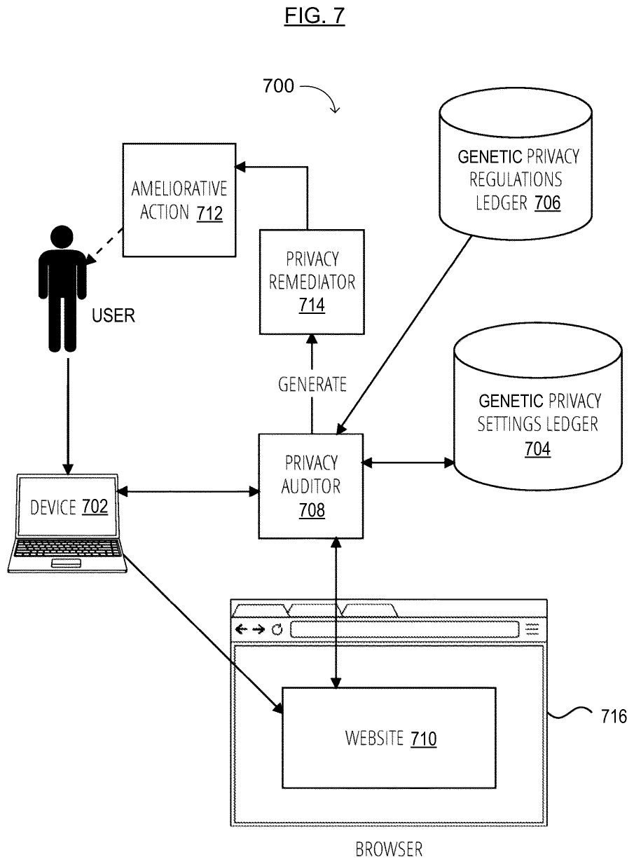

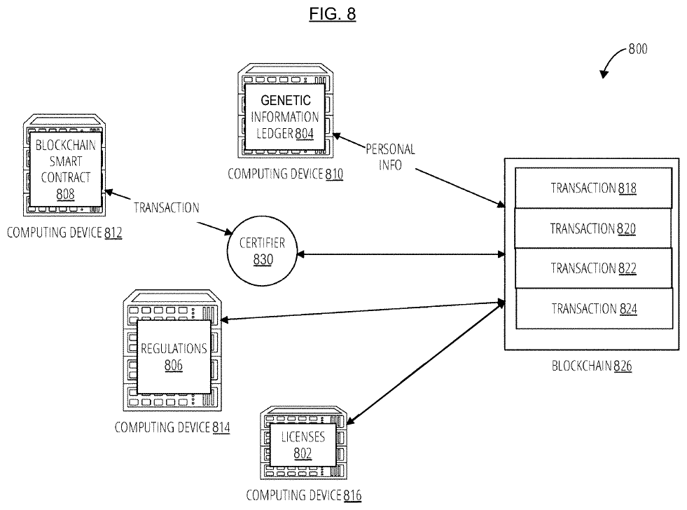

Referring now to FIG. 7, a genetic information management system 700 may comprise an electronic communication device 702, a genetic information privacy settings ledger 704, a genetic information privacy regulations ledger 706, a privacy auditor 708, a website 710, an ameliorative action 712, a privacy remediator 714, and a browser 716. The web portal 330 (FIG. 3) may comprise the electronic communication device 702 and the browser 716.

A universal privacy settings/opt-in/opt-out client (a “universal client”) allows a user to connect to an application program interface (API) for one or more different sites that have the user's data. The universal client orchestrates curation of privacy settings and overall opting in or out of any sites that the user selects or that are provided by default. It allows the user to select total or partial opt-ins or opt-outs where the user has granular control when they may wish to allow some uses of data and access to data, but restrict others. When a user is calibrating their privacy and data settings, a company or site or distributed application may provide reasons and incentives for the user to allow access to certain data (see the discussion of FIG. 6 above). This allows users to have simultaneous global control over their data/information, while enabling the user to receive compensation and/or services for the use of their data/information, thus allowing companies to have access to better data.

The user may retain global control over the entered data and enable others to access and/or use certain of the data by maintaining a universal profile with a privacy policy, which may be applied to company or owner privacy policies. In some cases, the system may automatically resolve conflicts between the privacy policy and the company/owner privacy policy or policies. Common settings across sites may have a unified view, and unique settings per site may be labeled with a site identifier. This allows the data and privacy settings to remain consistent across sites where common data and settings are used and uniquely where required by individual sites.

The user may authenticate the privacy system 700 into web sites and decentralized services and authorize its access to the sites utilizing the user's credentials. Where blockchain IDs are used, the privacy system may similarly operate on behavior of the user. For example, after the user has installed the system's user portion(s) (e.g., using electronic communication device 702), a user may use a mobile device or computer (e.g., similar or identical to device 702) to go to a site 710 or decentralized service, such as Facebook, Steemit, or STR-ID (Lewes, DE). The first time the user does this, the system 700 may automatically generate a pop-up window or notification and ask the user for their settings, allowing the system to auto-configure based on the user's online behavior. This allows the user greater freedom to use the most efficient software for their purpose, since the user is not forced to access sites 710 through the system 700, which runs parallel to, or in the user's browser 716. The system 700 may run in the background (like a daemon) and monitor sites 710 unobtrusively.

The system 700 may then see that the user accessed a site 710 or decentralized service that had in the past contained the user's data. The system 700 may inquire how the user would like to have their data managed on that site 710. The system 700 may allow the user to also configure when the system 700 is running. The system 700 may, for example, allow the user to toggle the system off and on, or allow the user to set specific instances or sites which should be explicitly included or excluded (i.e. “whitelisted” or “blacklisted”), or allow the user to “suspend” protection if desired.

When that site 710 or decentralized service is accessed in the future, the system 700 enforces the privacy settings in the ledger 704 through the browser 716 or interface used to access that site 710. Otherwise, the site 710 may automatically (re) configure the user's profile. For example, a user's Facebook profile may be automatically configured to reflect the user's preferences for Facebook's website (or decentralized service).

The system 700 may synchronize with privacy settings in the genetic information privacy settings ledger 704 that the user has changed manually to resolve and/or approve conflicts. When the system 700 connects to the privacy settings or opt-in/opt-out settings of the site 710 or the decentralized service, the system 700 may evaluate such settings to see if any changes were made. The system 700 may access a site 710 or decentralized service through an API or more directly through “web scraping” and may employ the user's ID and other information (e.g., in the ledger 704) to gain access. The system 700 may utilize an intermediary to analyze the settings and do a manual translation until the system 700 can gain access to the site 710. The system 700 may be configured with a country's or other jurisdiction's (e.g., the European Union's) privacy laws (e.g., recorded in the personal/genetic information privacy regulations ledger 706) and may monitor information on websites and decentralized services for compliance with both the user's settings as well as the privacy laws of that jurisdiction.

In the system 700, the privacy auditor 708 may scan websites 710 for the user's information. For example, the privacy auditor 708 may configure the browser 716 with a concept filter (not shown), and the browser 716 may then analyze data on the site 710. The browser 716 may then detect information on the site 710 that is not congruent with the genetic privacy settings ledger 704 and/or the genetic privacy regulations ledger 706. The browser 716 may then notify the privacy auditor 708, and the privacy auditor 708 may then notify the privacy remediator 714. An alert generator (e.g., in the device 702 and/or browser 716) receives the notification and generates an ameliorative action 712. The ameliorative action 712 may comprise, for example, accessing the incongruent data on the site 710 (e.g., via an API) and correcting the incongruence, or populating and transmitting a form cease-and-desist letter to the host and/or owner of the site 710 or decentralized service. The browser 716 may monitor and “crawl” websites for the user's information and/or the plant's, animal's, or living product's identification information, and may access and monitor such information on distributed applications, such as blockchain-based distributed services, sites, and applications.

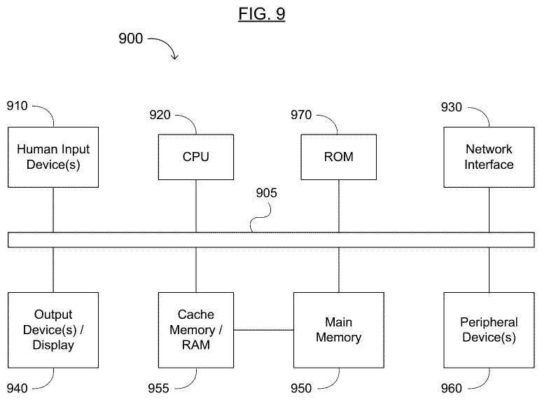

FIG. 8 shows an exemplary distributed blockchain storage network 800 comprising a computing device 810 storing a genetic information ledger 804 (e.g., similar or identical to ledger 620, FIG. 6), a computing device 812 storing a blockchain smart contract 808, a computing device 814 storing regulations 806 (e.g., in the form of a genetic information privacy regulations ledger), a computing device 816 storing one or more licenses 802, a certifier 830, and a plurality of transactions 818, 820, 822 and 824 in a blockchain 826. The computing device 810 transmits contents of genetic information ledger 804 (as needed) to the blockchain 826 and vice versa. The computing device 812 transmits the blockchain smart contract 808 (as needed) to the blockchain 826 via the certifier 830 and vice versa.

The computing device 814 records the regulations 806 on the blockchain 826, and the computing device 816 records the licenses 802 on the blockchain 826. The licenses 802 may include certain identification information of the user, owner, and the plant, animal or living product, as well as any licenses or third-party permissions necessary to share the genetic identification information with others. The licenses 802, regulations 806, blockchain smart contract 808 and personal information ledger 804 may be recorded on the blockchain 826 as the transaction 818, the transaction 820, the transaction 822, and the transaction 824, respectively. The blockchain 826 may be distributed on or among the computing devices 810, 812, 814 and 816.

### An Exemplary System

Similar to the present method, the present system may be divided into two parts or sections: a genetic testing part or section, and a digitalization/identification information management part or section. Referring back to FIG. 3, the genetic testing part or section of an exemplary system 300 comprises one or more DNA testing kits 310, and the digitalization/identification information management part or section of the exemplary system 300 comprises a DNA or genetic information digitization system 320, a web portal 330, and a mobile application 340, as described herein.

In one example, the DNA test kit 310 may comprise a DNA home sample collection kit. DNA sample collection kits may be commercially available from companies that produce DNA analysis kits, such as Thermo Fisher, Promega, and Qiagen, but can also be readily assembled. A typical kit 310 includes a tube or sample cup with a cap or lid for collecting the DNA sample, and detailed written instructions for the user to properly collect the sample and return it to the test facility (e.g., analysis lab). Detailed procedures for forensic DNA sample collection are well-known and widely available (see, e.g., Tan, E., “Sample Collection System for DNA Analysis of Forensic Evidence: Towards Practical, Fully-Integrated STR Analysis,” NIJ Award 2008-DN-BX-K010, Document No. 236826, December 2011, National Criminal Justice Reference Service, Rockville, MD; www.geneticprofiles.com/procedure/; and blog.puritanmedproducts.com/how-to-collect-dna-evidence, among others). Optionally, the kit 310 includes a swab (e.g., for procuring a saliva or other body fluid sample from the animal, etc.) or an absorbent paper or cotton pad (e.g., for absorbing a blood sample from the animal or for protecting a sample of a designated tissue or structure from the plant, etc.). The kit 310 typically also contains an envelope or box for shipping the sample to lab or test facility for analysis, and a container (e.g., a box or envelope) in which all of the kit components are placed.

The mobile application 340 may be installed on an electronic communication device 200 (FIG. 2A) such as a smartphone. The smartphone may further include features such as an on-off button or switch and an application-closing/switching and/or screen-changing button, among others.

The web portal 330 may be included on a webpage (e.g., 710, FIG. 7) accessible through a browser (e.g., 716). For example, registration may be conducted using the web portal 330, which can be accessed by the smartphone or on an alternative electronic communication device 900 as shown in FIG. 9. The electronic communication device 900 may be in the form of a personal computer, workstation, tablet computer, personal digital assistant, or the like.

FIG. 9 shows a basic architecture for the electronic communication device 900, including components such as one or more human input devices 910, a central processing unit (CPU) 920, a network interface 930, an output and/or display device 940, main memory 950, cache memory and/or random access memory (RAM) 955, one or more peripheral devices 960, and a read-only memory (ROM) 970. These components communicate with each other over one or more busses 905. The architecture of the electronic communication device 900 is largely conventional.

For example, the human input device(s) 910 may comprise a keyboard (e.g., a stand-alone or virtual keyboard), a mouse, a microphone (working together with speech recognition software stored in the main memory 950 and executed by the CPU 920), finger print reader, facial recognition system, etc. The network interface 930 may enable communications between the electronic communication device 900 and a home network, an intranet, a data and/or voice network, and/or the Internet, and may be wired or wireless. The output and/or display device 940 may comprise a monitor, display screen, television, one or more speakers, etc. The main memory 950 may comprise a magnetic or nonvolatile (e.g., flash) hard drive, configured to store software programs, data, user preferences, etc. The cache memory and/or random access memory (RAM) 955 may temporarily store recently used programs, routines or subroutines of programs, data, etc. for more facile use of such data, programs and (sub) routines. The peripheral device(s) 960 may comprise devices such as a printer, an external memory, speakers, a wireless receiver (e.g., from other devices such as a keyboard, mouse, etc.), a camera, a smartphone or tablet computer, etc. The read-only memory (ROM) 970 may store information and programs that generally cannot be erased or reprogrammed, such as device booting or start-up information, disk operating system (DOS) software, device configuration settings, etc.

The invention may be implementable in any of a variety of different types of blockchain networks. In particular, the present system may be implemented using a public blockchain network, a private blockchain network, a permissioned blockchain network, a consortium blockchain, or a combination thereof. Examples of such blockchain networks and the functions and transactions that they carry out are shown in FIGS. 5-8 and discussed in some detail above.

A private blockchain network, similar to a public blockchain network, is a decentralized peer-to-peer network, with the difference that one organization governs the network. (In a public blockchain network, no one organization or entity governs the network.) The organization that governs the private blockchain network controls who receives permission to participate in the network, executes a consensus protocol, and maintains the shared ledger. Alternatively, the organization that governs the private blockchain network may also control who executes a consensus protocol and maintains the shared ledger. Depending on the use case, this can significantly boost trust and confidence between participants. A private blockchain can be run behind a firewall and be hosted on-premises.

Businesses that set up a private blockchain often set up a permissioned blockchain network. Public blockchain networks may also be permissioned blockchain networks. This may place restrictions on (1) who may participate in the network and (2) the transactions in which certain participants may participate. Participants need to obtain an invitation or permission to join a permissioned blockchain network.

Multiple organizations can share the responsibilities of maintaining a blockchain. These organizations (which may be pre-selected) determine who may submit transactions or access the data stored in a ledger. A consortium blockchain network is ideal when all participants need to be permissioned and have a shared responsibility for the blockchain.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

What is claimed is:

1. A method of obtaining and controlling non-human genetic identification information, comprising:
a) providing identifying information of a plant, a non-human animal, or a living plant or animal product to a secure website using a first electronic communication device;
b) taking a genetic material-containing sample from the plant, the non-human animal, or the living plant or animal product;
c) providing the genetic material-containing sample to a genetic material analysis facility;
d) analyzing the genetic material at a plurality of loci to produce a genetic identity for the plant, the non-human animal, or the living plant or animal product;
e) recording the identifying information and the genetic identity in a blockchain ledger; and
f) enabling a user to display on a second electronic communication device a code corresponding to the genetic identity, wherein the first and second electronic communication devices are a same device or different devices.

2. The method of claim 1, wherein said identifying information comprises at least two of a name, an owner name, an owner address, and a photograph of the plant, the non-human animal, or the living plant or animal product.

3. The method of claim 1, further comprising encrypting the identifying information and the genetic identity of the plant, the non-human animal, or the living plant or animal product prior to recording the identifying information and the genetic identity in the blockchain ledger.

4. The method of claim 1, wherein the first and second electronic communication devices are independently selected from a smart phone, a smart watch, a personal computer, a tablet computer, and a work station.

5. The method of claim 1, wherein the plant, the non-human animal, or the living plant or animal product is the non-human animal.

6. The method of claim 5, wherein taking the genetic material-containing sample from the non-human animal comprises collecting saliva from the non-human animal in a vial or tube, swabbing an inner surface of the non-human animal's mouth or nose, or pricking/puncturing the non-human animal's skin and collecting one or more drops of the non-human animal's blood on a swab or piece of absorbent paper.

7. The method of claim 5, wherein analyzing the genetic material comprises extracting DNA from the genetic material, and analyzing the DNA by short tandem repeat (STR) analysis, whole genomic and/or next generation DNA sequencing, mitochondrial DNA (mtDNA) sequencing, or analysis of single-nucleotide polymorphisms (SNPs).

8. The method of claim 5, wherein the non-human animal is a pet.

9. The method of claim 8, wherein the pet is a dog or a cat.

10. The method of claim 5, wherein the non-human animal is a livestock animal.

11. The method of claim 10, wherein the livestock animal is a bovine, a horse or a sheep.

12. The method of claim 1, wherein the plant, the non-human animal, or the living plant or animal product is the plant.

13. The method of claim 12, wherein analyzing the genetic material comprises extracting DNA from the genetic material, and analyzing the DNA by analysis of single-nucleotide polymorphisms (SNPs), genotyping by sequencing (GBS), next-generation sequencing (NGS), polymerase chain reaction (PCR)-based sequencing, or 3^{rd} generation sequencing (TGS).

14. The method of claim 1, wherein the plant, the non-human animal, or the living plant or animal product is an asexually reproduced plant.

15. The method of claim 1, further comprising (i) certifying or confirming that the genetic material has been collected or (ii) certifying or confirming that a sample collector has authority to collect the genetic material.

16. The method of claim 1, wherein providing the genetic material-containing sample to the genetic material analysis facility comprises shipping the genetic material-containing sample to the genetic material analysis facility in an envelope, sleeve, tube or box.

17. The method of claim 1, further comprising allowing the user to access entries in the blockchain ledger containing the identifying information and the genetic identity.

18. The method of claim 1, wherein a registrant provides the identifying information of the plant or the non-human animal to the secure website, and the method further comprises enabling the registrant to authorize third parties to access the code on a third electronic communication device, wherein third electronic communication device is identical to, same as, or different from one or both of the first and second electronic communication devices.

19. The method of claim 1, further comprising accessing the code using one of the first and second electronic communication devices.

20. The method of claim 1, further comprising authenticating an identity or the identifying information of the plant, the non-human animal, or the living plant or animal product using the genetic identity.
